(19) United States
(12) Patent Application Publication
(10) Pub. No.: US 2025/0108154 A1
(43) Pub. Date: Apr. 3, 2025

(54) RESTRICTIVE VERSUS LIBERAL RATE OF EXTRACORPOREAL VOLUME REMOVAL EVALUATION IN ACUTE KIDNEY INJURY

(71) Applicants: UNIVERSITY OF PITTSBURGH - OF THE COMMONWEALTH SYSTEM OF HIGHER EDUCATION, Pittsburgh, PA (US); MAYO FOUNDATION FOR MEDICAL EDUCATION AND RESEARCH, Rochester, MN (US)

(72) Inventors: Raghavan Murugan, Wexford, PA (US); Paul M. Palevsky, Sewickley, PA (US); Kianoush Kashani, Rochester, MN (US)

(21) Appl. No.: 18/834,468

(22) PCT Filed: Feb. 2, 2023
(86) PCT No.: PCT/US2023/012204

§ 371 Date: Jul. 30, 2024

Related U.S. Application Data
(60) Provisional application No. 63/305,790, filed on Feb. 2, 2022.

(51) Int. Cl. A61M 1/16; G16H 10/60

(52) U.S. Cl. CPC A61M 1/1613; A61M 1/1611; G16H 10/60

(57) ABSTRACT

The invention pertains to treating kidney disease and systems and methods for determining the rate of net fluid removal to be used during CKRT. According to the invention, determining the rate of net fluid removal includes calculating a predicted body weight of the patient based on patient's height, determining a net fluid removal rate from the patient in volume/mass/time based on the predicted body weight of the patient and a restrictive or a liberal strategy, calculating a total rate of continuous intravenous fluids infused into the patient in volume/mass/time, and adding the total rate of continuous intravenous fluids infused to the net fluid removal rate. The net fluid removal rate is used to set the continuous dialysis machine to remove a corresponding fluid volume from the patient during CKRT.

### CROSS-REFERENCE TO RELATED APPLICATIONS

This application claims priority under 35 U.S.C. § 119 (e) to U.S. Provisional Patent Application Ser. No. 63/305,790, filed Feb. 2, 2022, entitled “RESTRICTIVE VERSUS LIBERAL RATE OF EXTRACORPOREAL VOLUME REMOVAL EVALUATION IN ACUTE KIDNEY INJURY,” the content of which is herein incorporated by reference.

### STATEMENT OF GOVERNMENT INTEREST

This invention was made with government support under grant #DK128100, awarded by the National Institutes of Health (NIH). The government has certain rights in the invention.

### FIELD OF THE INVENTION

The invention pertains to treating kidney disease and, in particular, systems and methods for determining the rate of net fluid removal used during continuous kidney replacement therapy (CKRT) (i.e., continuous dialysis).

### BACKGROUND OF THE INVENTION

Acute kidney injury (AKI) and fluid overload occur in more than 50% of intensive care unit (ICU) admissions. About 5-6% of ICU patients with AKI receive kidney replacement therapy (i.e., dialysis) for removing fluid. The hospital mortality rates in this population exceed 50%. However, the optimal fluid removal rate during CKRT remains uncertain.

Currently, no electronic decision support tools are available to help critical care practitioners determine the rate at which fluid should be removed during CKRT. Thus, there is wide variation in clinical practice, and clinicians typically remove fluid at an arbitrary rate. This results in complications such as hypotension or cardiac arrhythmias when fluid is removed too fast or fluid overload complications when fluid is removed too slowly from the patient.

“Net ultrafiltration (UF_{NET}),” also known as net fluid removal during kidney replacement therapy, has been used in treating fluid overload among critically ill patients with AKI for over seven decades. The UF_{NET} is the net volume of fluid removed from the patient by the dialysis machine after discounting fluids administered (e.g., replacement fluids and dialysate) via the dialysis machine for the purpose of conducting the dialysis. The “net UF_{NET} rate” is the rate at which the intravascular fluid volume is removed from the patient adjusted for the patients' body weight and unit time (i.e., milliliters/kilogram/hour). The optimal rate of fluid removal (i.e., UF_{NET} rate) remains uncertain, the complications mentioned above (e.g., hypotension and cardiac arrhythmias) occur frequently, and more than 40% of patients die. Observational studies in critically ill patients receiving CKRT show that both slower and faster UF_{NET} rates are associated with an increased risk of death compared to moderate UF_{NET} rates. Slower UF_{NET} rates, compared with faster rates, increase exposure to fluid overload and organ edema. Faster UF_{NET} rates, compared with slower rates, are associated with hemodynamic instability, hypotension, and ischemic organ injury. Therefore, slower and faster rates are associated with mortality compared with moderate UF_{NET} rates in observational studies. Thus, there is a need to determine whether the UF_{NET} rate-mortality relationship is causal.

Additionally, there is a need to establish the feasibility of maintaining patients in a restrictive UF_{NET} rate strategy during treatment with CKRT and assess whether a restrictive UF_{NET} rate strategy embracing a “slow and steady” approach to fluid removal is associated with fewer complications, including cardiac arrhythmias, hypotension, and death, compared with a more liberal “sprint and pause” strategy among critically ill patients.

As fluid is removed from the intravascular space during extracorporeal ultrafiltration, vascular refill occurs due to fluid shifting from the extravascular and interstitial spaces into the intravascular space. When fluid removal is higher than the vascular refill rate, intravascular hypovolemia results in hypotension, decreased organ perfusion, and ischemic injury. Although there are patient-related (e.g., comorbid conditions, reduced vasomotor tone) and other dialysis-related factors (e.g., reduced plasma osmolality due to solute clearance) that contribute to hemodynamic instability, several studies indicate that there is a direct relationship between higher UF_{NET} rate, a process of care variable, and subsequent risk of hypotension and mortality.

Clinical assessment of intravascular volume is the holy grail of hemodynamic management in critically ill patients during ultrafiltration. Conventionally used hemodynamic parameters such as blood pressure, central venous pressure, and pulmonary artery occlusion pressures are insensitive to early changes in intravascular volume. Although dynamic parameters such as pulse pressure variation, stroke volume variation, IVC collapsibility, and passive leg raising are used to predict fluid responsiveness in critically ill patients, these technologies have several pitfalls. First, assessing pulse pressure variation and stroke volume variation to predict fluid responsiveness requires that the patient is mechanically ventilated, not on low tidal volume ventilation, sedated, and not spontaneously breathing. Second, IVC collapsibility and passive leg-raising tests are not feasible as they cannot be done continuously. Third, while these technologies have been validated for predicting fluid responsiveness in critically ill patients, their validity for predicting intradialytic hypotension during fluid removal in critically ill patients is unknown. Finally, other technologies, such as hematocrit monitoring and bioimpedance analysis, used in outpatients undergoing hemodialysis, have not been validated in critically ill patients for fluid removal. Thus, the current assessment of intravascular volume during ultrafiltration is mostly clinically based on surrogate measures such as blood pressure, fluid balance, and physical examination of the patient (e.g., capillary refill).

Accordingly, there is a need to develop and standardize net fluid removal based on clinical information that is easily and conveniently derivable from the patient to reduce practice variation and improve clinical outcomes.

### SUMMARY OF THE INVENTION

In one aspect, the invention provides a method for determination of net fluid removal from a patient during continuous kidney replacement therapy. The method includes calculating a predicted body weight of the patient based on patient's height, selecting one of a restrictive strategy comprising 0.5 to 1.5 mL/kg/h and a liberal strategy comprising 2.0 to 5.0 mL/kg/h; based on the predicted body weight and the selected restrictive or liberal strategy, determining the net fluid removal rate; calculating a total rate of continuous intravenous fluids infused into the patient in volume/mass/time; adding the total rate of continuous intravenous fluids infused to the net fluid removal rate to determine the final net fluid removal rate; and setting the final net fluid removal rate on a CKRT machine to remove fluid.

The predicted body weight may be calculated according to the equations:

$$predicted body weight ( kg . ) = 45.5 + 2.3 [ height ( in . ) - 60 ] for female patients , \\ and \\ predicted body weight ( kg . ) = 50 + 2.3 [ height ( in . ) - 60 ] for male patients .$$

The total rate of fluids infused into the patient may be selected from the group consisting of intravenous fluids, medications, blood, plasma, and combinations thereof.

The step of determining the net fluid removal rate may be according to a restrictive strategy. The restrictive strategy can have initiation and maintenance phases. The initiation phase can include an initial removal rate of 0.5 mL/kg/hr. If the patient tolerates the initial removal rate, the fluid removal rate can be gradually increased by 0.5 mL/kg/h. If the patient tolerates the gradually increased fluid removal rate, the maintenance phase can be implemented, having a net removal rate of 0.5 to 1.5 mL/kg/br.

The step of determining the net fluid removal rate may be according to a liberal strategy. The liberal strategy can have initiation and maintenance phases. The initiation phase can include an initial removal rate of 0.5 mL/kg/hr. If the patient tolerates the initial removal rate, the fluid removal rate can be gradually increased by 0.5 mL/kg/h. If the patient tolerates the gradually increased fluid removal rate, the maintenance phase can be implemented, having a net removal rate of 2.0 to 5.0 mL/kg/hr.

The method can include entering into a web-based calculator the height of the patient to calculate the predicted body weight and continuous intravenous fluid infusion into the patient to calculate the net fluid removal rate per hour. The web-based calculator may include an algorithm that calculates and determines steps.

In another aspect, the invention provides a method of controlling a continuous dialysis machine. The method includes determining a net fluid removal rate in volume/mass/time, including calculating a predicted body weight of the patient based on patient's height; determining a net fluid removal rate from the patient in volume/mass/time based on the predicted body weight of the patient and selection of a restrictive strategy or a liberal strategy; calculating a total rate of intravenous fluids infused into the patient in volume/mass/time; and adding the total rate of intravenous fluids infused to the net fluid removal rate; setting the continuous dialysis machine at the final net fluid removal rate to remove fluid from the patient.

In yet another aspect, the invention provides a continuous dialysis system that including an electronic tool structured and configured to determine a net fluid removal rate component in volume/mass/time, that includes receiving first information indicative of predicted body weight of a patient; receiving second information indicative of a total rate of continuous intravenous fluids infused into the patient in volume/mass/time; and determining a net fluid removal rate from the patient in volume/mass/time based on the predicted body weight of the patient and selection of a liberal strategy or a restrictive strategy; and adjusting the net fluid removal rate based on the total rate of continuous intravenous fluids infused; and an algorithm, in communication with the electronic tool, is structured and configured to provide output comprising the patient's net fluid removal rate to be set on the CKRT machine based on the predicted body weight, the total rate of continuous intravenous fluids infused into the patient, and the selection of the restrictive strategy or the liberal strategy.

### DETAILED DESCRIPTION

As used herein, the singular form of “a,” “an,” and “the” include plural references unless the context clearly dictates otherwise.

The disclosed concept will now be described, for purposes of explanation, in connection with numerous specific details to provide a thorough understanding of the subject invention. It will be evident, however, that the disclosed concept can be practiced without these specific details and without departing from the spirit and scope of this innovation.

CKRT treatment is used for volume control in critically ill patients and provides continuous ultrafiltration over 24 hours in patients with severe fluid overload. However, the optimal ultrafiltration rates in critically ill patients with AKI treated with CKRT are uncertain. Accordingly, the disclosed concept includes a method of net fluid removal from a patient during CKRT, e.g., UF_{NET}. The method includes calculating the patients' predicted body weight (PBW) to determine and set the UF_{NET} rate. The patient's height is indicative of the PBW. Therefore, according to the following equations, PBW is estimated using a gender-specific calculator based on measuring patients' height (from heel to crown).

$$Males : PBW ( Kilograms ) = 50 + 2.3 [ height ( inches ) - 60 ] \\ Females : PBW ( Kilograms ) = 45.5 + 2.3 [ height ( inches ) - 60 ]$$

PBW is shown closely to approximate ideal body weight (IBW) in males and females because it is free of confounding by fluid overload and catabolismdue to critical illness and other measurement errors by nursing staff and is precisely determined from patient height accurately measured by trained clinicians. Measured body weight (MBW) and actual body weight is not used because precise premorbid MBW is unknown in most critically ill patients. Weight documented in EMR during prior hospitalization cannot be used as a surrogate for premorbid weight because of confounding by an underlying illness. Additionally, index hospital admission weight is likely to be confounded by the underlying condition that led to hospitalization (e.g., dehydration from sepsis may result in underestimation, and fluid overload from the underlying worsening congestive heart may result in overestimation of MBW). In addition, retrospective collection of hospital and ICU admission weights are prone to measurement errors and confounded by daily fluid intake and output, which are inaccurately documented in hospital wards. Furthermore, following initiation of UF_{NET}, the patient MBW is likely to fluctuate widely with decreasing weight over time due to the catabolic nature of many ICU illnesses or increasing due to fluid administration and consequent fluid overload.

The invention method determines a UF_{NET} rate for the patient during CKRT according to a restrictive UF_{NET} rate strategy or a liberal UF_{NET} rate strategy. The restrictive UF_{NET} rate strategy includes a net fluid removal rate from 0.5 to 1.5 mL/kg/h, and the alternative liberal UF_{NET} strategy includes a net fluid removal rate from 2.0 to 5.0 mL/kg/h. The restrictive and liberal strategies include initiation and maintenance phases.

For the restrictive UF_{NET} rate strategy, the initiation phase consists of setting an initial UF_{NET} rate at 0.5 mL/kg/h based on the patient's PBW; if tolerated by the patient, the UF_{NET} rate is gradually increased to 0.5 mL/kg/h each hour up to a maximum of 1.5 mL/kg/h; if tolerated by the patient, the maintenance phase is then implemented, which consists of maintaining the UF_{NET} rate from 0.5 to 1.5 mL/kg/h. Accordingly, in certain embodiments, for an 80-kg patient who is not receiving any continuous intravenous infusions, the UF_{NET} rate is initiated at 40 mL/h (i.e., 80 kg×0.5 mL/kg/h) and gradually increased by 40 mL/h to a maximum rate of 120 mL/h (i.e., 80 kg×1.5 mL/kg/h) as tolerated. The UF_{NET} rate is then maintained from 40 to 120 mL/h.

For the liberal UF_{NET} rate strategy, the initiation phase consists of setting an initial UF_{NET} rate at 0.5 mL/kg/h based on the patient's PBW; if tolerated by the patient, the UF_{NET} rate is gradually increased 0.5 mL/kg/h each hour; if tolerated by the patient, the maintenance phase is then implemented, which consists of maintaining the UF_{NET} rate from 2.0 to 5.0 mL/kg/h. Accordingly, in certain embodiments, for an 80-kg patient who is not receiving any continuous intravenous infusions, the UF_{NET} rate is initiated at 40 mL/h (i.e., 80 kg×0.5 mL/kg/h) and gradually increased by 40 mL/h to a maximum rate of 400 mL/h (i.e., 80 kg×5.0 mL/kg/h) as tolerated. The UF_{NET} rate is then maintained from 160 (i.e., 80 kg×2.0 mL/kg/h) to 400 mL/h.

Restrictive UF_{NET} rates have been found to reduce the risk of cardiac arrhythmias and short- and long-term mortality, and kidney replacement therapy (KRT) dependence in observational studies. In addition, in certain embodiments, restrictive UF_{NET} rates result in fewer hypotensive episodes, an overall net negative balance, and improved patient outcomes due to reduced risk of hypotension and subsequent fluid administration.

The restrictive strategy provides one or more of the following benefits. First, a restrictive strategy allows more time for a vascular refill and reduces blood pressure variability and hypotensive episodes, which are associated with ischemic organ injury and mortality. Second, by preventing hypotensive episodes, the restrictive UF_{NET} rate will likely reduce the need for subsequent interventions such as completely discontinuing UF_{NET}, fluid administration, or starting or increasing the vasopressor dose. Both discontinuing UF_{NET} and fluid administration will offset any potential benefit of UF_{NET} and increase the risk of fluid overload. Third, restrictive UF_{NET} rates preserve myocardial blood flow and prevent episodes of cardiac arrhythmias. Fourth, a restrictive UF_{NET} rate reduces the workload and burden on nursing staff due to decreased number of interventions, e.g., titrations, required to treat intradialytic hypotension.

The restrictive UF_{NET} rate strategy also provides one or more of the following considerations. Restrictive UF_{NET} rate strategy may be associated with longer tissue exposure to fluid overload and may increase the time to achieving cuvolemia. Prolonged exposure to fluid overload may impair kidney recovery, prolong the duration of kidney replacement therapy, or increase ventilator dependence. However, the risks of prolonged exposure to fluid overload associated with restrictive UF_{NET} rate strategy must be balanced against the risk of ischemic organ injury due to hypotensive episodes and blood pressure variability associated with faster and more liberal UF_{NET} rates used in clinical practice.

The liberal strategy provides one or more of the following benefits. The liberal UF_{NET} rate based on hemodynamics may result in better and earlier volume control, including achieving daily negative fluid balance and, overall, less positive cumulative fluid balance as documented in observational studies. By varying UF_{NET} across a range of UF_{NET} rates, the liberal UF_{NET} strategy affords more flexibility to clinicians for rapid fluid removal for the treatment of fluid overload. The restrictive UF_{NET} rate strategy also provides one or more of the following considerations. Liberal UF_{NET} rate strategy may be associated with a rapid and unpredictable decline in intravascular volume. Intravascular hypovolemia, in turn, reduces cardiac preload. Decreased preload is associated with lower cardiac output and hypotension. Moreover, faster and more frequent titration of UF_{NET} increases the workload for the clinicians, causes poor compliance with closer monitoring of hemodynamics, and increases subsequent interventions for the treatment of hypotensive episodes, including bolus fluid administration offsetting potential benefits of rapid fluid removal.

Whereas, in certain embodiments, liberal UF_{NET} rates result in reduced exposure to fluid overload, earlier liberation from mechanical ventilation, and lower complications from fluid overload.

The UF_{NET} rate, i.e., according to a restrictive or liberal strategy, is adjusted to account for the total rate of continuous intravenous fluids infused into the patient during ultrafiltration, e.g., in mL/hr. The total rate of continuous intravenous fluids infusion is added to the UF_{NET} rate to obtain a final net fluid removal rate. The continuous intravenous fluids infused include, but are not limited to, intravenous fluids, medications, blood, plasma, and combinations thereof.

In certain embodiments, during the initiation phase of a restrictive or liberal strategy, a method of treating the patient and corresponding data is as follows.
Step 1: enter the total patient continuous intravenous fluids infusion in a UF_{NET} rate calculator for the current hour.
Step 2: enter whether the patient is in the liberal or restrictive strategy into the calculator.
Step 3: set the calculator recommended UF_{NET} rate on the CKRT machine.

In certain embodiments, during the maintenance phase, there is no requirement to enter the calculator or change the CKRT rate every hour unless there is a need.

As described herein, a novel method of net fluid removal from a patient during KRT includes calculating a PBW of the patient based on patient's height, determining a net fluid removal rate from the patient in volume/mass/time based on the PBW of the patient and selection of a restrictive or a liberal strategy; calculating a total rate of continuous intravenous fluids infused into the patient in volume/mass/time; adding the continuous intravenous fluids infusion rate to the net fluid removal rate; and setting this adjusted (e.g., final rate) on the CKRT machine, irrespective of restrictive or liberal UF_{NET} strategy.

For example, in a patient with PBW of 80 kilograms, if the continuous intravenous patient fluid infusion rate is 50 mL/h after adding all intravenous fluid infusions in that hour, to deliver a UF_{NET} rate of 1.0 mL/kg/h (restrictive strategy), the fluid removal rate would be set at 130 mL/h [(80 kg×1.0 mL/kg/h)+50 mL/h] in the CKRT machine. This would ensure the patient receives a UF_{NET} rate of 1.0 mL/kg/h. In contrast, if that 50 mL/h continuous intravenous fluid infusion is stopped in the patient, the CKRT machine fluid removal rate would be reset at 80 mL/kg/h [(80 kg×1.0 mL/kg/h)+0 mL/h] to deliver a UF_{NET} rate of 1.0 mL/kg/h.

The invention provides an electronic decision support tool to help critical care practitioners determine the rate at which fluid should be removed during CKRT. The electronic decision support tool, e.g., software, assists, for example, ICU nurses in determining the correct rate of fluid removal and setting it in the dialysis machines. The net fluid removal rate is used to set the continuous dialysis machine to remove a corresponding fluid volume from the patient during CKRT.

The algorithm allows the net fluid removal rate calculation to be set on a continuous dialysis machine. The algorithm is based on the patient's PBW and the fluid infusion rate as described above. The algorithm is incorporated in a computer (e.g., iPad, laptop, or desktop computer) application (“app”) and/or incorporated into the continuous dialysis machine software by manufacturing companies.

In certain embodiments, accurate calculation of the net fluid removal rate includes the use of PBW, current hour patient intravenous (IV) fluids, and the use of a web application mounted on an iPad or desktop computer. In certain embodiments, the disclosed concept includes a fluid removal rate calculator.

According to the invention, it is typical for a clinical practitioner to enter patient information/data into the web application. The information/data includes, but is not limited to, the height of the patient for calculation of PBW, the volume of continuous intravenous fluid infused into the patient (mL/h) in the current hour, and the desired net fluid removal rate based on restrictive or liberal method, to determine the final rate of net fluid removal be set on the CKRT machine.

Thus, the invention provides a novel method of net fluid removal that includes an electronic tool that can be structured and configured to determine a net fluid removal rate component in volume/mass/time using an algorithm. The algorithm uses the PBW and the continuous patient intravenous infusion in the hour to determine the net fluid removal rate to be set on the CKRT machine.

As aforementioned, UF_{NET}, during KRT, is used to treat fluid overload among critically ill patients with AKI. Therefore, understanding the UF_{NET} rate-outcome relationship in acutely ill patients is critical for four reasons: a) to ensure that the provision of current care is safe, b) to design interventions to reduce mortality, c) to develop evidence-based clinical practice guidelines, and d) to implement quality measures during treatment with CKRT.

For patients with >5% fluid overload and treated with CKRT and IHD, the inventors found that UF_{NET} rates<20 mL/kg/day, compared with rates>25 mL/kg/day, were associated with increased risk-adjusted mortality. Of the patients treated with CKRT, hourly UF_{NET} rates<0.5 mL/kg/h compared with rates>1.0 mL/kg/h were also associated with death. These findings suggest that minimum UF_{NET} rates of >20 mL/kg/day or >1.0 mL/kg/h using CKRT are associated with a lower risk of death among patients with fluid overload. Further, UF_{NET} rates<35 mL/kg/day compared with ≥35 mL/kg/day were also associated with the risk of major adverse kidney events. These findings suggest that a minimum rate of at least 1.0 mL/kg/h is associated with reduced mortality compared with slower rates.

Any reference signs placed between parentheses shall not be construed as limiting the claim in the claims. The word “comprising” or “including” does not exclude the presence of elements or steps other than those listed in a claim. In a device claim enumerating several means, several of these means may be embodied by the same hardware item. The word “a” or “an” preceding an element does not exclude the presence of a plurality of such elements. In any device claim enumerating several means, several of these means may be embodied by the same item of hardware. The fact that certain elements are recited in mutually different dependent claims does not indicate that these elements cannot be combined.

Although the invention has been described in detail for illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is understood that the present invention contemplates that, to the extent possible, one or more features of any embodiment can be combined with one or more features of any other embodiment.

### EXAMPLES

### Background

A goal was to determine whether a restrictive UF_{NET} rate strategy is associated with lower mortality than a liberal UF_{NET} rate strategy in critically ill patients with AKI. A hypothesis is that a restrictive UF_{NET} rate strategy embracing a “slow and steady” approach to fluid removal is associated with fewer complications, including cardiac arrhythmias, hypotension, and death, compared with a more liberal “sprint and pause” strategy among critically ill patients.

### Study Conducted

Using the Randomized Evaluation of Normal versus Augmented Level (RENAL) of the Renal Replacement Therapy trial cohort, the association of the UF_{NET} rate with outcomes was examined. Of 1,434 patients, the 90-day mortality among patients who received UF_{NET} rate>1.75 vs. 1.01-1.75 vs.<1.01 mL/kg/h was: 48.6% vs. 39.2% vs. 44.9%; P=0.01, respectively. Using Gray model, UF_{NET} rates>1.75 mL/kg/h compared with rates 1.01-1.75 mL/kg/h (adjusted HR range, 1.44-1.77, P=0.004) and rates<1.01 mL/kg/h (adjusted HR range, 1.51-1.66; P=0.01) was associated with lower survival. Every 0.5 mL/kg/h increase in UF_{NET} rate was associated with a 7% increased odds of death (adjusted OR, 1.07; 95% CI, 1.00-1.15). Using a joint model, longitudinal increase and variation in UF_{NET} rates over time were also associated with the risk of death (13=0.056; P<0.001). UF_{NET} rates of >1.75 mL/kg/h were also associated with an increased risk of cardiac arrhythmias requiring treatment (36.8% vs. 30.8%; P=0.08).

After accounting for competing risk of death, UF_{NET} rates>1.75 mL/kg/h compared with UF_{NET} rates, 1.01-1.75 mL/kg/h (cause-specific adjusted HR, 0.79, 95% CI, 0.66-0.95) and UF_{NET} rates, <1.01 ml/kg/h (adjusted HR, 0.69, 95% CI, 0.56-0.85) was associated with longer dependence on KRT.

Also investigated was whether the daily fluid balance was a mediator of the relationship between UF_{NET} rate and mortality, with baseline day one fluid balance as moderator. It was found that a more negative daily fluid balance attenuated the harmful mortality effect of the high UF_{NET} (>1.75 mL/kg/h) rate group compared with the moderate (1.01-1.75 mL/kg/h) and low (<1.01 mL/kg/h) UF_{NET} rate groups. However, despite this attenuation, the high UF_{NET} rate (>1.75 ml/kg/h) group remained significantly and directly associated with higher mortality compared with the moderate UF_{NET} rate group (average direct effect, 1.10, 95% CI, 1.04-1.16).

Also examined was the association between the UF_{NET} rate within the first 48 hours of use of CKRT and hospital mortality in an independent cohort of 347 critically ill patients. UF_{NET} rates>1.75 ml/kg/h compared with rates<1.01 ml/kg/h (adjusted HR range, 1.27-4.18, P=0.03) was associated with 28-day mortality. In a subsequent mediation analysis, it was found that this higher risk of death was not mediated by fluid balance, blood pressure, vasopressors, or electrolytes, implying that higher UF_{NET} rates may have a direct causal effect on the risk of death.

Using the University of Pittsburgh Medical Center (UPMC) ICU database of patients with >5% fluid overload and treated with CKRT and IHD (n=1,075), the association of UF_{NET} rate over 24 hours and 1-year mortality was evaluated. It was found that the UF_{NET} rates<20 mL/kg/day, compared with rates>25 mL/kg/day, were associated with increased risk-adjusted mortality. Of the CKRT subgroup, hourly UF_{NET} rates<0.5 mL/kg/h compared with rates>1.0 mL/kg/h were also associated with death. These findings suggest that minimum UF_{NET} rates of >20 mL/kg/day or >1.0 mL/kg/h using CKRT are associated with a lower risk of death among patients with fluid overload. Using Mayo Clinic data, UF_{NET} rates<35 mL/kg/day compared with ≥35 mL/kg/day were also associated with the risk of major adverse kidney events. These studies suggest that a minimum rate of at least 1.0 mL/kg/h is associated with reduced mortality compared with slower rates.

Using cluster analysis in RENAL, the probability of harm associated with UF_{NET} rates>1.75 mL/kg/h was 99.6% com-pared with UF_{NET} rates of 1.01-1.75 mL/kg/h and 32.5% compared with UF_{NET} rates<1.01 mL/kg/h among the subgroup of severely ill patients who had sepsis, metabolic acidosis, organ edema, those treated with mechanical ventilation and vasopressors. The probability of harm associated with UF_{NET} rates between 1.01-1.75 mL/kg/h compared with the rates<1.01 mL/kg/h was only 0.2%. Of patients who are hemodynamically unstable with cardiovascular sequential organ failure as-sessment (SOFA) score of 3 or more, both UF_{NET} rates>1.75 mL/kg/h and rates<1.01 mL/kg/h were associated with increased mortality compared with rates 1.01-1.75 mL/kg/h.

In a survey of 80 countries, two-thirds of practitioners (71%, regional range, 55%-95.5%) reported using CKRT for volume management. In the U.S., the reported initial median UF_{NET} rate prescription was 100 mL/h (IQR, 78-200), and the maximum rate was 285 mL/h (200-341) for hemodynamically stable patients and 51 mL/h (IQR, 25-100) for hemodynamically unstable patients. For an average 80-kg patient, a UF_{NET} volume of 285 mL/h would be equivalent to a rate of 3.56 mL/kg/h, and 341 mL/h would equal a rate of 4.26 mL/kg/h in hemodynamically stable patients. These data suggest that the UF_{NET} rates used in the liberal group are within the range of clinical practice. More than 80% of practitioners believed a protocol-based fluid removal would be useful.

Of 5,967 days of CKRT treatment, the per-patient median UF_{NET} volume was 102.4 (IQR, 50-180) mL/hour. The UF_{NET} rates at 1% and 99% percentiles were 0-511.92 mL/h. After adjusting for weight, the median UF_{NET} rate was 1.34 (0.63-2.39) mL/kg/h. The 1% and 99% percentile UF_{NET} rates were 0-6.55 mL/kg/h. These data suggest that the UF_{NET} rates selected for restrictive and liberal groups are within current clinical practice in the U.S.

What is claimed is:

1. A method for determination of net fluid removal from a patient during continuous kidney replacement therapy, comprising:
calculating a predicted body weight of the patient based on patient's height;
selecting one of a restrictive strategy comprising 0.5 to 1.5 mL/kg/h and a liberal strategy comprising 2.0 to 5.0 mL/kg/b;
based on the predicted body weight and the selected restrictive or liberal strategy, determining the net fluid removal rate;
calculating a total rate of continuous intravenous fluids infused into the patient in volume/mass/time;
adding the total rate of continuous intravenous fluids infused to the net fluid removal rate to determine the final net fluid removal rate; and
setting the final net fluid removal rate on a CKRT machine to remove fluid.

2. The method of claim 1, wherein the predicted body weight is calculated according to the equations:
$$predicted body weight ( kg . ) = 45.5 + 2.3 [ height ( in . ) - 60 ] for female patients , and \\ predicted body weight ( kg . ) = 50 + 2.3 [ height ( in . ) - 60 ] for male patients .$$

3. The method of claim 1, wherein the total rate of fluids infused into the patient is selected from the group consisting of intravenous fluids, medications, blood, plasma, and combinations thereof.

4. The method of claim 1, wherein determining the net fluid removal rate is according to a restrictive strategy.

5. The method of claim 4, wherein the restrictive strategy has initiation and maintenance phases.

6. The method of claim 5, wherein the initiation phase comprises an initial removal rate of 0.5 mL/kg/hr.

7. The method of claim 6, wherein if the patient tolerates the initial removal rate, the fluid removal rate is gradually increased by 0.5 mL/kg/h.

8. The method of claim 7, wherein if the patient tolerates the gradually increased fluid removal rate, the maintenance phase is implemented, having a net removal rate of 0.5 to 1.5 mL/kg/hr.

9. The method of claim 1, wherein determining the net fluid removal rate is according to a liberal strategy.

10. The method of claim 9, wherein the liberal strategy has initiation and maintenance phases.

11. The method of claim 10, wherein the initiation phase comprises an initial removal rate of 0.5 mL/kg/hr.

12. The method of claim 11, wherein if the patient tolerates the initial removal rate, the fluid removal rate is gradually increased by 0.5 mL/kg/h.

13. The method of claim 12, wherein if the patient tolerates the gradually increased fluid removal rate, the maintenance phase is implemented, having a net removal rate of 2.0 to 5.0 mL/kg/hr.

14. The method of claim 1, further comprises entering into a web-based calculator the height of the patient to calculate the predicted body weight and continuous intravenous fluid infusion into the patient to calculate the net fluid removal rate per hour.

15. The method of claim 1, wherein the web-based calculator comprises an algorithm that calculates and determines steps.

16. A method of controlling a continuous dialysis machine comprising:
determining a net fluid removal rate in volume/mass/time, comprising:
calculating a predicted body weight of the patient based on patient's height;
determining a net fluid removal rate from the patient in volume/mass/time based on the predicted body weight of the patient and selection of a restrictive strategy or a liberal strategy;
calculating a total rate of intravenous fluids infused into the patient in volume/mass/time; and
adding the total rate of intravenous fluids infused to the net fluid removal rate; and
setting the continuous dialysis machine at the final net fluid removal rate to remove fluid from the patient.

17. A continuous dialysis system comprising:
an electronic tool structured and configured to determine a net fluid removal rate component in volume/mass/time, comprising:
receiving first information indicative of predicted body weight of a patient;
receiving second information indicative of a total rate of continuous intravenous fluids infused into the patient in volume/mass/time; and
determining a net fluid removal rate from the patient in volume/mass/time based on the predicted body weight of the patient and selection of a liberal strategy or a restrictive strategy; and
adjusting the net fluid removal rate based on the total rate of continuous intravenous fluids infused; and
an algorithm, in communication with the electronic tool, is structured and configured to provide output comprising the patient's net fluid removal rate to be set on the CKRT machine based on the predicted body weight, the total rate of continuous intravenous fluids infused into the patient, and the selection of the restrictive strategy or the liberal strategy.
